(19) United States
(12) Patent Application Publication
(10) Pub. No.: US 2022/0323201 A1
(43) Pub. Date: Oct. 13, 2022

(54) IMPLANT FOR ATTACHING A TENDON OR A LIGAMENT TO A HARD TISSUE

(71) Applicant: GARY A. ZWICK, TRUSTEE OF THE EVEREST TRUST UTA APRIL 20, 2017, Cleveland, OH (US)

(72) Inventors: George J. PICHA, Brecksville, OH (US); James PRICE, Stow, OH (US); Gregory CAUSEY, Erie, CO (US)

(21) Appl. No.: 17/753,498

(22) PCT Filed: Sep. 10, 2020
(86) PCT No.: PCT/US20/50178

§ 371 Date: Mar. 4, 2022

Related U.S. Application Data
(60) Provisional application No. 62/898,726, filed on Sep. 11, 2019.

(51) Int. Cl. A61F 2/08

(52) U.S. Cl. CPC A61F 2/0811; A61F 2220/0016

(57) ABSTRACT

An implant for attaching a tendon or ligament to a hard tissue is provided. The implant includes a head, a first prong, a second prong, first pillars for contacting a hard tissue, first slots to be occupied by the hard tissue, second pillars for contacting a tendon or ligament, and second slots to be occupied by the tendon or ligament. The implant has a Youngs modulus of elasticity of at least 3 GPa, a first surface ratio of the sum of the volumes of the first slots to the sum of the volumes of the first pillars and the first slots of 0.40:1 to 0.90:1, and a second surface ratio of the sum of the volumes of the second slots to the sum of the volumes of the second pillars and the second slots of 0.60:1 to 0.98:1. The second surface ratio is greater than the first surface ratio.

### FIELD OF THE INVENTION

The invention relates to implants for attaching a tendon or a ligament to a hard tissue, and more particularly to implants for attaching a tendon or a ligament to a hard tissue that comprise a head, a first prong, a second prong, first pillars for contacting a hard tissue, and second pillars for contacting a tendon or a ligament.

### BACKGROUND OF THE INVENTION

Tendons are bands of dense fibrous connective tissue that attach muscle to bone. Ligaments are bands of fibrous tissue that bind joints together and connect articular bones and cartilages to facilitate movement.

It is estimated that about 300,000 tendon and ligament repair surgeries are performed in the United States each year (Yang et al. (2013), Birth Defects Res C Embryo Today, 99:203-222). Common repair surgeries include rotator cuff repair, patellar tendon repair, and anterior cruciate ligament reconstruction. Unfortunately, tendon and ligament reattachment surgeries often fail due to failure of regeneration of enthesis, corresponding to a specialized transitional tissue that connects tendon and ligament to bone by a gradual change in structure, composition, and mechanical behavior, thereby effectively transferring stress from tendon and ligament to bone and vice versa despite the tendon and ligament being compliant and the bone being stiff (Liu et al. (2010), Journal of Orthopaedic Surgery and Research, 5:59).

Entheses occur in two types. The first type, direct insertions, also termed fibrocartilaginous entheses, are composed of four zones: tendon or ligament, uncalcified fibrocartilage, calcified fibrocartilage, and bone. Tendon and ligament fibers are passed directly into bone cortex in small surface areas of the bone, including deep fibers attached to bone at right angles or tangentially. Examples include anterior cruciate ligament, Achilles tendon, patellar tendon, rotator cuff, and femoral insertion of medial collateral ligament. The second type, indirect insertions, also termed fibrous entheses, has no fibrocartilaginous interface. For indirect insertions, the tendon or ligament passes along the bone surface obliquely, inserts into the bone periosteum, and connects over larger surface areas of the bone than for direct insertions. Examples include tibial insertion of the medial collateral ligament and insertion of the deltoid tendon into the humerus.

Conventional approaches for surgical reattachment of tendons and ligaments to bones involve performing tendon or ligament grafts. In some approaches, a tendon or ligament, typically obtained from another part of a patient, is attached to an implant or a bone plug and inserted into a bone tunnel, with suturing to secure the graft to the bone. In some approaches, a tendon is passed through holes in a bone, and then an interference screw is inserted, forcing the tendon against bone to fix the tendon in place. In some approaches, a ligament is looped through holes in a bone, without use of an implant, a bone plug, or an interference screw.

Following tendon and ligament reattachment surgeries, tendon/ligament and bone healing occurs through formation of fibrovascular scar tissue, not reestablishment of enthesis (Apostolakos et al (2014), Muscles Ligaments and Tendons Journal, 4:333-342). This apparently contributes to failures of the surgeries.

Conventional hard-tissue implants include implants designed to promote ingrowth of hard tissue based on forming a tissue/implant interface in which the implant forms a continuous phase and the tissue forms a discontinuous phase, e.g. based on the implant having a concave and/or porous surface into which the hard tissue can grow, and designed to have add-on surface modifications, e.g. modifications added based on sintering.

For example, Van Kampen et al., U.S. Pat. No. 4,608,052, discloses an implant for use in a human body having an integral attachment surface adapted to permit ingrowth of living tissue. The implant surface is defined by a multiplicity of adjacent, generally concave surface parts having intersecting, generally aligned rims defining an inner attachment surface portion and by a multiplicity of spaced posts projecting from the inner attachment surface. Van Kampen also discloses that implants have been provided with porous surfaces, as described in U.S. Pat. Nos. 3,605,123, 3,808,606, and 3,855,638.

Also for example, J. D. Bobyn et al, 150 Clinical Orthopaedics & Related Research 263 (1980), discloses that a pore size range of approximately 50 to 400 μm provided an optimal or maximal fixation strength (17 MPa) in the shortest time period (8 weeks) with regard to cobalt-base alloy implants with powder-made porous surfaces. Specifically, implants were fabricated based on coating cylindrical rods of cast cobalt-base alloy with cobalt base alloy powder in four particle size ranges. The particle size ranges were as follows: 25 to 45 μm; 45 to 150 μm; 150 to 300 μm; and 300 to 840 μm. The corresponding pore size ranges of the particles were as follows: 20 to 50 μm; 50 to 200 μm; 200 to 400 μm; and 400 to 800 μm, respectively. The particles were then bonded to the rods based on sintering. All implants were manufactured to have a maximal diameter of 4.5 mm and a length of 9.0 mm. The implants were surgically inserted into holes in dog femurs and bone ingrowth was allowed to proceed. After varying periods of time (4, 8, or 12 weeks), the maximum force required to dislodge the implants was determined. Implants with a pore size lower than 50 μm yielded relatively low fixation strengths at all time points, while implants with a pore size higher than 400 μm exhibited relatively high scatter with regard to fixation strengths, thus indicating that a pore size range of approximately 50 to 400 μm provided an optimal or maximal fixation strength.

Conventional hard-tissue implants also include implants having surface texturing, e.g. raised portions and indented portions, barbs, and/or pillars, to promote an interference fit between the implants and adjacent bone, to make it difficult to withdraw the implants from hard tissue, or to more effectively mechanically anchor at an early date or affix into adjoining hard tissue.

For example, Tuke et al., U.K. Pat. Appl. No. GB2181354A, discloses an orthopedic implant having at least one surface area, integral with the adjacent portion of the implant and adapted in use to contact bone. The surface area has a finely patterned conformation composed of a plurality of raised portions separated from each other by indented portions. The indented portions are of a width and depth to allow bone penetration thereinto in use to promote an interference fit between the implant and adjacent bone in the region of the patterned area.

Also for example, Amrich et al., U.S. Pat. No. 7,018,418, discloses implants having a textured surface with microrecesses such that the outer surface overhangs the microrecesses. In one embodiment, unidirectional barbs are produced in the surface that can be inserted into bone or tissue. The directional orientation of the barbs is intended to make it difficult to withdraw from the bone or tissue.

Also for example, Picha, U.S. Pat. No. 7,556,648, discloses a spinal implant, i.e. an implant for use in fusing and stabilizing adjoining spinal vertebrae, including a hollow, generally tubular shell having an exterior lateral surface, a leading end, and a trailing end. The exterior surface includes a plurality of pillars arranged in a non-helical array. Each pillar has a height of 100 to 4,500 μm and a lateral dimension at the widest point of 100 to 4,500 μm. The exterior surface also has a plurality of holes therethrough to permit bone ingrowth therethrough.

Unfortunately, interfaces of hard tissue and hard-tissue implants in which the hard tissue is in a discontinuous phase may be susceptible to stress shielding, resulting in resorption of affected hard tissue, e.g. bone resorption, over time. Also, addition of surface texturing to implants by sintering can result in the surface texturing occupying an excessive volume of corresponding hard tissue/implant interfaces, leaving insufficient space for hard tissue. In addition, spinal implants are designed to perform under conditions relevant to spine, i.e. compression, rotational shear, and vertical shear, with the compression being essentially constant, the rotational shear being intermittent, and the vertical shear being rare, rather than conditions relevant to other hard tissues such as long bone, maxillary bone, mandibular bone, and membranous bone, i.e. load bearing conditions, including compression and tension, varying across the hard tissue and across time, and intermittent rotational and vertical shear.

Picha et al., U.S. Pat. No. 8,771,354, discloses hard-tissue implants including a bulk implant, a face, pillars, and slots. The hard-tissue implant has a Young's modulus of elasticity of at least 10 GPa, has a ratio of (i) the sum of the volumes of the slots to (ii) the sum of the volumes of the pillars and the volumes of the slots of 0.40:1 to 0.90:1, does not comprise any part that is hollow, and does not comprise any non-pillar part extending to or beyond the distal ends of any of the pillars. The hard-tissue implants can provide immediate load transfer upon implantation and prevent stress shielding over time, thus promoting hard-tissue remodeling and growth at the site of implantation. The interface can have a continuous phase corresponding to the hard tissue and a discontinuous phase corresponding to the hard-tissue implant.

A need exists for implants for attachment of a tendon or a ligament to a hard tissue that account for tendon/ligament and bone healing occurring through formation of fibrovascular scar tissue, to effectively transfer stress from tendon and ligament to bone and vice versa despite the tendon and ligament being compliant and the bone being stiff.

### BRIEF SUMMARY OF THE INVENTION

An implant for attaching a tendon or a ligament to a hard tissue is provided. The implant comprises:

(a) a head having an upper surface, a lower surface, and an aperture between the upper surface and the lower surface;

(b) a first prong extending from the lower surface of the head, and having a first prong outer surface, a first prong inner surface, and a first prong tip;

(c) a second prong extending from the lower surface of the head, and having a second prong outer surface, a second prong inner surface, and second prong tip;

(d) first pillars for contacting a hard tissue, the first pillars being distributed on the first prong outer surface and the second prong outer surface across an area of at least 20 mm². on each, and extending distally therefrom, and each first pillar being integral to the first prong or the second prong, having a distal end, having a transverse area of (100×100) to (2,000×2,000) μm², and having a height of 100 to 2,000 μm;

(e) first slots to be occupied by the hard tissue, the first slots being defined by the first pillars and each first slot having a width of 100 to 2,000 μm as measured along the shortest distance between adjacent first pillars;

(f) second pillars for contacting a tendon or a ligament, the second pillars being distributed on the first prong inner surface and the second prong inner surface across an area of at least 20 mm². of each, and extending distally therefrom, and each second pillar being integral to the first prong or the second prong, having a distal end, having a transverse area of (200×200) to (4,000×4,000) μm², and having a height of 100 to 5,000 μm; and

(g) second slots to be occupied by the tendon or the ligament, the second slots being defined by the second pillars and each second slot having a width of 400 to 4,000 μm as measured along the shortest distance between adjacent second pillars.

The implant has (1) a Young's modulus of elasticity of at least 3 GPa, (2) a ratio of (i) the sum of the volumes of the first slots to (ii) the sum of the volumes of the first pillars and the volumes of the first slots (“first surface ratio”) of 0.40:1 to 0.90:1, and (3) a ratio of (i) the sum of the volumes of the second slots to (ii) the sum of the volumes of the second pillars and the volumes of the second slots (“second surface ratio”) of 0.60:1 to 0.98:1.

The second surface ratio is greater than the first surface ratio.

In some embodiments, the implant is made of one or more materials selected from implantable-grade polyaryletherketone that is essentially unfilled, implantable-grade polyetheretherketone, implantable-grade polyetherketoneketone, titanium, stainless steel, cobalt-chromium alloy, titanium alloy, Ti-6Al-4V titanium alloy, Ti-6Al-7Nb titanium alloy, ceramic material, silicon nitride (Si3N4), implantable-grade composite material, implantable-grade polyaryletherketone with filler, implantable-grade polyetheretherketone with filler, implantable-grade polyetheretherketone with carbon fiber, or implantable-grade polyetheretherketone with hydroxyapatite. Also, in some embodiments, the implant is made of one or more hard tissues selected from human hard tissue, animal hard tissue, autologous hard tissue, allogenic hard tissue, xenogeneic hard tissue, human cartilage, animal cartilage, human bone, animal bone, cadaver bone, or cortical allograft. Also, in some embodiments, the implant is made of one or more materials selected from resin for rapid prototyping, SOMOS® NanoTool non-crystalline composite material, SOMOS® 9120 liquid photopolymer, SOMOS® WaterShed XC 11122 resin, ACCURA® XTREME™ White 200 plastic, or ACCURA® 60) plastic.

In some embodiments, the first prong and the second prong extend perpendicularly from the lower surface of the head.

In some embodiments, the first prong is tapered toward the first prong tip, and the second prong is tapered toward the second prong tip.

In some embodiments, the first pillars extend in a uniform direction. Also, in some embodiments, the first pillars are perpendicular to the first prong outer surface and the second prong outer surface. Also, in some embodiments, the first pillars are angled toward the head.

In some embodiments, the second pillars extend in a uniform direction. Also, in some embodiments, the second pillars on the first prong inner surface are angled toward the first prong tip and the second pillars on the second prong inner surface are angled toward the second prong tip.

In some embodiments, the transverse area of each first pillar is (200×200) μm² to (1,000×1,000) μm².

In some embodiments, the height of each first pillar is 200 to 900 μm.

In some embodiments, one or more of the first pillars have dimensions that differ from those of other first pillars, such that the transverse areas and/or heights, and thus volumes, of the one or more first pillars differ from those of the other first pillars.

In some embodiments, the width of each first slot is 200 to 900 μm.

In some embodiments, the transverse area of each second pillar is (400×400) μm² to (2,000×2,000) μm².

In some embodiments, the height of each second pillar is 200 to 4,000 μm.

In some embodiments, one or more of the second pillars have dimensions that differ from those of other second pillars, such that the transverse areas and/or heights, and thus volumes, of the one or more second pillars differ from those of the other second pillars.

In some embodiments, the width of each second slot is 500 to 3,000 μm.

In some embodiments, the head has a head diameter at a widest portion of the head, the implant has first prong length from the head to the first prong tip, the implant has a second prong length from the head to the second prong tip, the implant has a ratio of the first prong length to the head diameter of 2.0 to 10, and the implant has a ratio of the second prong length to the head diameter of 2.0 to 10.

In some embodiments, the head has a head diameter of 4 to 20 mm at a widest portion of the head.

In some embodiments, the implant has a first prong length of 8 to 40 mm from the head to the first prong tip, and the implant has a second prong length of 8 to 40 mm from the head to the second prong tip.

In some embodiments, one or more of the head, the first prong, the first pillars, the second prong, or the second pillars are non-porous. Also, in some embodiments, one or more of the head, the first prong, the first pillars, the second prong, or the second pillars are porous.

Also provided is a method of use of the implant for attaching a tendon or a ligament to a hard tissue in an individual in need thereof. The method comprises steps of:

(1) preparing a bone tunnel in a bone of the individual;

(2) attaching a tendon or a ligament to the implant such that the tendon or ligament is in contact with the second pillars of the implant; and

(3) inserting the implant into the bone tunnel;

The method results in attaching the tendon or the ligament to the bone of the individual.

In some embodiments, the preparing of the bone tunnel comprises drilling a hole in the bone.

In some embodiments, the attaching of the tendon or the ligament to the implant comprises pulling the tendon or the ligament through the aperture, thereby putting the tendon or ligament in contact with the second pillars of the implant.

In some embodiments, the attaching of the tendon or the ligament to the implant comprises piercing the tendon or the ligament with the second pillars, thereby putting the tendon or ligament in contact with the second pillars of the implant.

In some embodiments, the inserting of the implant into the bone tunnel comprises pressing the implant into the bone tunnel.

In some embodiments, the method does not comprise use of a suture or an adhesive to secure the tendon or the ligament to the implant.

### BRIEF DESCRIPTION OF THE DRAWINGS

These and other features, aspects, and advantages of the present disclosure are better understood when the following detailed description is read with reference to the accompanying drawings, in which:

FIG. 1 is a first perspective view of an implant for attaching a tendon or a ligament to a hard tissue as disclosed herein;

FIG. 2 is a second perspective view of the implant of FIG. 1;

FIG. 3 is a first side view of the implant of FIG. 1;

FIG. 4 is a second side view of the implant of FIG. 1;

FIG. 5 is a top view of the implant of FIG. 1;

FIG. 6 is a bottom view of the implant of FIG. 1; and

FIG. 7 is side view of the implant of FIG. 1 to which a tendon has been pressed onto second pillars of the implant.

### DETAILED DESCRIPTION

As set forth in the figures, example implants for attaching a tendon or a ligament to a hard tissue are provided. The implants provide advantages, including for example that the implant can provide a surface for attachment of a tendon or ligament, protect the tendon or ligament upon placement of the implant in a hard tissue, promote hard-tissue remodeling and growth of the hard tissue at the site of implantation, and hold the tendon or ligament stable following implantation to allow formation of a fibrovascular scar tissue that can effectively transfer stress from tendon or ligament to bone and vice versa. Without wishing to be bound by theory, it is believed that the resulting interfaces of the implant, the tendon or ligament, and the hard tissue can withstand substantial yield/elongation and load before failure.

This is because the implants provide an interface with a hard tissue at the first prong outer surface and the second prong outer surface that can have a continuous phase corresponding to the hard tissue and a discontinuous phase corresponding to the implant. The hard tissue can also make up at least 40% of the volume of the interface, and the product of the Young's modulus of elasticity of the hard tissue and the volume of the tissue and the product of the Young's modulus of elasticity of the implant and the volume of the first pillars of the implant can be well matched. Thus, the interface can exhibit mechanical properties similar to those of the bulk hard tissue adjacent to the interface. Also, the first pillars may be pressed into the hard tissue, potentially eliminating micro-motion and migration of the implant over time, accommodating torque, and/or eliminating the need for adhesives such as cement or grout to hold the implant in place. In addition, the implants may promote rich vascularization of the hard tissue of the interface, enhancing wound healing, providing nutritional support, accelerating healing, remodeling, and integration of the hard tissue, and limiting the potential for infection of the hard tissue. Rapid or immediate integration of the hard tissue into the space between the first pillars of the implant may also prevent detrimental cellular reactions at the interface, such as formation of fibrous tissue, seroma, or thrombosis.

This also is because the implants provide an interface with the tendon or ligament at the first prong inner surface and the second prong inner surface that allows for attachment of the tendon or ligament to the implant, for example based on piercing of the tendon or ligament by the second pillars in a discrete area of limited size. The tendon or ligament also can make up at least 60% of the volume of the interface, minimizing damage to cell structure of the tendon or ligament between the second pillars, while the second pillars protect the tendon or ligament from damage upon implantation of the implant in the hard tissue and hold the tendon or ligament stable following implantation. In addition, the combination of rich vascularization of adjacent hard tissue and stability of the tendon or ligament may allow formation of a fibrovascular scar tissue that can effectively transfer stress from tendon or ligament to bone and vice versa.

As used herein, the term “implant for attaching a tendon or a ligament to a hard tissue” means an implant suitable for attaching a tendon or a ligament to a hard tissue based on implantation in a hard tissue. Exemplary hard tissues suitable for implantation of the implants include bones such as humerus, e.g. for rotator cuff repair, patella or tibial tubercle, e.g. for patellar tendon repair, and femur and tibia, e.g. for anterior cruciate ligament reconstruction, among other bones.

As used herein, the term “pillar” means a projection that extends distally from a surface of the implant, that is not in direct physical contact with any other pillars or other parts of the implant other than the surface, and that is for contacting a hard tissue or a tendon or ligament. Because a pillar is not in direct physical contact with any other pillars or other parts of the implant other than the surface, upon implantation no pillar forms a continuous phase within the resulting interface of the hard tissue, tendon, or ligament and the implant.

A pillar can have a transverse area, i.e. an area of a cross-section taken relative to a vertical axis along which the pillar extends distally from the surface of the implant, of, for example, (i) (100 μm×100 μm) to (2,000 μm×2,000 μm), i.e. 1.0×10⁴ μm² to 4.0×10⁶ μm², (ii) (200 μm×200 μm) to (1,000 μm×1,000 μm), i.e. 4.0×10⁴ μm² to 1.0×10⁶ μm², (iii) (250 μm×250 μm) to (1,000 μm×1,000 μm), i.e. 6.3×10⁴ μm² to 1.0×10⁶ μm², (iv) (300 μm×300 μm) to (500 μm×500 μm), i.e. 9×10⁴ μm² to 2.5×10⁵ μm², (v) (350 μm×350 μm) to (450 μm×450 μm), i.e. 1.2×10⁵ μm² to 2.0×10⁵ μm², or (vi) (395 μm×395 μm) to (405 μm×405 μm), i.e. 1.6×10⁵ μm². A pillar also can have a transverse area of, for example, (i) (200 μm×200 μm) to (4,000 μm×4,000 μm), i.e. 4.0×10⁴ μm² to 1.6×10⁷ μm², (ii) (400 μm×400 μm) to (2,000 μm×2,000 μm), i.e. 1.6×10⁵ μm² to 4.0×10⁶ μm², or (iii) (1,000 μm×1,000 μm) to (2,000 μm×2,000 μm), i.e. 1.0×10⁶ μm² to 4.0×10⁶ μm². Of note, the expression of transverse areas of pillars as squares of linear dimensions, e.g. (100 μm×100 μm), here and throughout this application, is for purposes of convenience only and is not intended to limit any pillars so described to square shapes, square transverse areas, or square cross-sections.

A pillar can have a pillar height, i.e. the height of the pillar from a surface of the implant to the distal end of the pillar, of, for example, 100 to 2,000 μm, 200 to 900 μm, 300 to 800 μm, or 400 to 600 μm. A pillar also can have a height of, for example, 100 to 5,000 μm, 200 to 4,000 μm, 300 to 3,000 μm, 400 to 2,000 μm, or 500 to 1,000 μm.

A pillar can have a volume, i.e. product of pillar transverse area and pillar height, of, for example (100 μm×100 μm×100 μm) to (2,000 μm×2,000 μm×2,000 μm), i.e. 1.0×10⁶ μm³ to 8×10⁹ μm³, among other volumes. A pillar also can have a volume of, for example (200 μm×200 μm×100 μm) to (4,000 μm×4,000 μm×5,000 μm), i.e. 4.0×10⁶ μm³ to 8.0×10¹⁰ μm³, among other volumes.

A pillar can have, as seen from a top view, a square shape, a rectangular shape, a herringbone shape, a circular shape, or an oval shape, respectively, or alternatively can have other polygonal, curvilinear, or variable shapes.

As used herein, the term “slot” means the spaces between the pillars. Accordingly, the pillars define the slots. The slots can have a slot height as defined by the pillars, of, for example, 100 to 2,000 μm, 200 to 900 μm, 300 to 800 μm, or 400 to 600 μm, or 100 to 10,000 μm, 100 to 8,000 μm, 100 to 7,000 μm, 100 to 6,000 μm, or 100 to 5,000 μm, among others. The slots can have a slot width as measured along the shortest distance between adjacent pillars of, for example, 100 to 2,000 μm, 150 to 1,000 μm, 200 to 700 μm, or 300 to 500 μm, or 400 to 4,000 μm, 500 to 3,000 μm, 600 to 2,000 μm, or 800 to 1,500 μm, among others. The slots have a volume corresponding to the volume of the space between the pillars.

As used herein, the term “pore” refers to a void space of less than 1,000 μm in size, i.e. having a diameter of less than 1,000 μm, on or below a surface, e.g. the surface of an implant. Pores can occur in a material naturally, e.g. based on a natural porosity of the material, or can be introduced, e.g. by chemical or physical treatment. Pores can be continuous with respect to each other, based on being interconnected with each other below a surface, or pores can be discontinuous, based on not being interconnected with each other below a surface. Pores can be sufficiently large to allow for migration and proliferation of osteoblasts and mesenchymal cells. Accordingly, for example, a porous surface is a surface that includes void spaces of less than 1,000 μm in size in the surface, whereas a non-porous surface is a surface that does not include such a void space.

As used herein, the term “interface” includes the product of implantation wherein the first pillars of the implant are contacting a hard tissue and the first slots of the implant are occupied, partially or completely, by the hard tissue. The term “interface” also includes the product of implantation wherein the second pillars of the implant are contacting a tendon or a ligament and the second slots of the implant are occupied, partially or completely, by the tendon or the ligament.

In some examples, e.g. immediately after implanting the implant with at least some penetration of the first pillars into the hard tissue and/or after at least some remodeling and growth of the hard tissue to partially fill in space between the implant and the hard tissue, the first pillars are contacting the hard tissue (e.g. at distal ends of the first pillars), and the first slots are partially occupied by the hard tissue. In other examples, e.g. immediately after implanting the implant with extensive penetration of the first pillars into the hard-tissue and/or after extensive remodeling and growth of the hard tissue to fill in all space between the implant and the hard tissue, the first pillars are contacting the hard tissue (e.g. at distal ends and lateral surfaces of the first pillars), and the first slots are completely occupied by the hard tissue. In other examples the first pillars contact the hard tissue over time, based on remodeling and growth of hard tissue in and around the first pillars, e.g. during healing.

As used herein, the term “continuous,” when used for example in reference to the hard-tissue of an interface, means that the hard tissue forms a single continuous phase, extending throughout and across the interface to each boundary of the interface. As used herein, the term “discontinuous,” when used for example in reference to the implant of an interface, means that the implant does not form such a single continuous phase.

Implant for Attaching a Tendon or a Ligament to a Hard Tissue

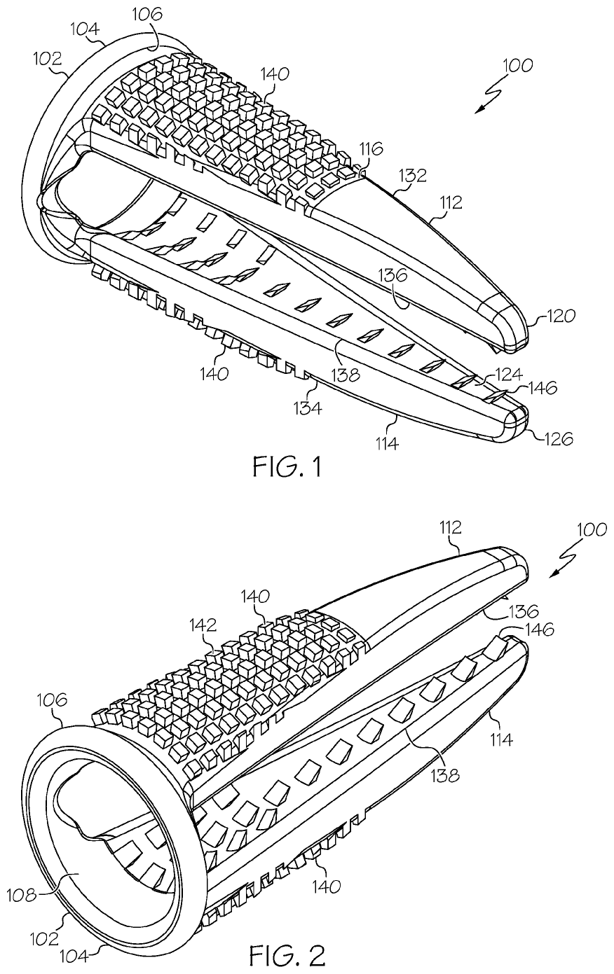

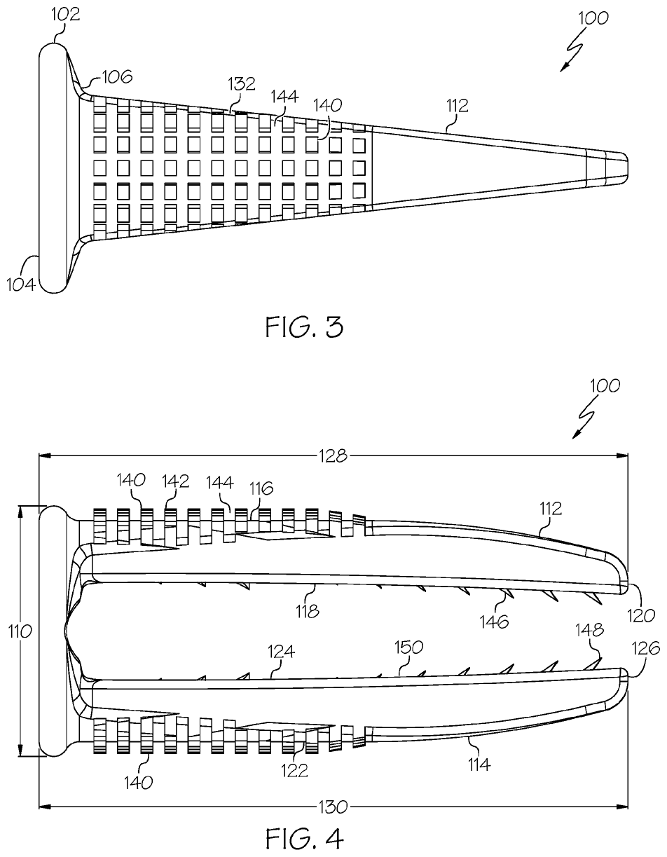

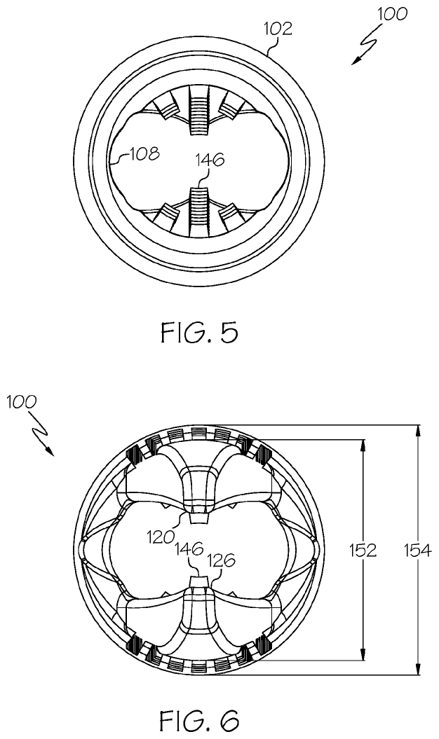

Considering the features of the implant for attaching a tendon or a ligament to a hard tissue in more detail, FIGS. 1-6 illustrate an embodiment of an implant 100 for attaching a tendon or a ligament to a hard tissue.

The implant 100 can be made from a material having a Young's modulus of elasticity, i.e. a tensile modulus of elasticity, of at least 3 GPa, as measured at 21° C. The implant 100 can be made, for example, from one or more materials such as implantable-grade polyaryletherketone that is essentially unfilled (such as implantable-grade polyetheretherketone or implantable-grade polyetherketoneketone), titanium, stainless steel, cobalt-chromium alloy, titanium alloy (such as Ti-6Al-4V titanium alloy or Ti-6Al-7Nb titanium alloy), ceramic material (such as silicon nitride (Si3N4)), or implantable-grade composite material (such as implantable-grade polyaryletherketone with filler, implantable-grade polyetheretherketone with filler, implantable-grade polyetheretherketone with carbon fiber, or implantable-grade polyetheretherketone with hydroxyapatite). Specific examples include (i) implantable-grade polyetheretherketone that is essentially unfilled, which has a Young's modulus of approximately 4 GPa, (ii) implantable-grade polyetheretherketone with filler, e.g. carbon-fiber-reinforced implantable-grade polyetheretherketone, which has a Young's modulus of elasticity of at least 18 GPa, (iii) titanium, which has a Young's modulus of elasticity of approximately 110 GPa, (iv) stainless steel, which has a Young's modulus of elasticity of approximately 200 GPa, (v) cobalt-chromium alloy, which has a Young's modulus of elasticity of greater than 200 GPa, or (vi) titanium alloy, which has a Young's modulus of elasticity of approximately 105-120 GPa, all as measured at 21° C. The implant 100 also can be made, for example, from one or more hard tissues such as a hard tissue obtained from a human or animal (such as autologous hard tissue, allogenic hard tissue, or xenogeneic hard tissue), human cartilage, animal cartilage, human bone, animal bone, cadaver bone, or cortical allograft. Such hard tissues obtained from a human or animal can have a Young's modulus of elasticity of, e.g. 4 to 18 GPa. Such hard tissues obtained from a human or animal can also be treated, in advance of implantation, to decrease or eliminate the capacity of the hard tissue to elicit an immune response in an individual upon implantation into the individual. The implant 100 also can be made, for example, from one or more materials such as resin for rapid prototyping, SOMOS® NanoTool non-crystalline composite material, SOMOS® 9120 liquid photopolymer, SOMOS® WaterShed XC 11122 resin, ACCURA® XTREME™ White 200 plastic, or ACCURA® 60) plastic. The implant 100 also can be made, for example, from one or more materials that are resorbable, such as polylactic acid or polycaprolactone, among others, in which case callus around bone and tendon at the site of implantation would gradually remove the polymer of the implant 100, with replacement by a patient's own tissue, which would be more analogous to a natural state. The implant 100 also can be made from further combinations of the above-noted materials and/or hard tissues. Accordingly, the implant 100 has a Young's modulus of elasticity of at least 3 GPa, for example 18 to 230 GPa, 18 to 25 GPa, 100 to 110 GPa, 190 to 210 GPa, 200 to 230 GPa, 105 to 120 GPa, or 4 to 18 GPa.

The implant 100 comprises a head 102 having an upper surface 104, a lower surface 106, and an aperture 108 between the upper surface 104 and the lower surface 106. The head 102 can have a generally cylindrical shape, although other shapes, e.g. conical shapes, or frustoconical shapes, may be used in further examples. In some examples the head 102 has a head diameter 110 of 4 to 20 mm at a widest portion of the head 102.

The head 102 can be made from one or more of the materials or hard tissues noted above with respect to the implant 100, e.g. one or more materials such as implantable-grade polyaryletherketone that is essentially unfilled (such as implantable-grade polyetheretherketone or implantable-grade polyetherketoneketone), titanium, stainless steel, cobalt-chromium alloy, titanium alloy (such as Ti-6Al-4V titanium alloy or Ti-6Al-7Nb titanium alloy), ceramic material (such as silicon nitride (Si3N4)), or implantable-grade composite material (such as implantable-grade polyaryletherketone with filler, implantable-grade polyetheretherketone with filler, implantable-grade polyetheretherketone with carbon fiber, or implantable-grade polyetheretherketone with hydroxyapatite), or e.g. one or more hard tissues such as a hard tissue obtained from a human or animal (such as autologous hard tissue, allogenic hard tissue, or xenogeneic hard tissue), human cartilage, animal cartilage, human bone, animal bone, cadaver bone, or cortical allograft, or e.g. one or more materials such as resin for rapid prototyping, SOMOS® NanoTool non-crystalline composite material, SOMOS® 9120 liquid photopolymer, SOMOS® WaterShed XC 11122 resin, ACCURA® XTREME™ White 200 plastic, or ACCURA® 60) plastic, or e.g. one or more materials that are resorbable, such as polylactic acid or polycaprolactone.

The head 102 can be porous or non-porous. For example, the head 102 can include one or more surfaces that are porous, and/or can be made from one or more materials that are porous. Such porous surfaces can include pores having diameters of, e.g. 1 to 900 μm, 100 to 800 μm, or 200 to 600 μm. Also for example, the head 102 can include only surfaces that are non-porous, and/or can be made only from one or more materials that are non-porous.

The implant 100 also comprises a first prong 112 and a second prong 114. The first prong 112 extends from the lower surface 106 of the head 102, and has a first prong outer surface 116, a first prong inner surface 118, and a first prong tip 120. The second prong 114 extends from the lower surface 106 of the head 102, and has a second prong outer surface 122, a second prong inner surface 124, and second prong tip 126.

Like the head 102, the first prong 112 and the second prong 114 can be made from one or more of the materials or hard tissues noted above with respect to the implant 100. The first prong 112 and/or the second prong 114 can be porous, e.g. including pores having diameters of, e.g. 1 to 900 μm, 100 to 800 μm, or 200 to 600 μm, or the first prong 112 and/or the second prong 114 can be non-porous.

In some examples the first prong 112 and the second prong 114 extend perpendicularly from the lower surface 106 of the head 102. In some examples the first prong 112 is tapered toward the first prong tip 120, and the second prong 114 is tapered toward the second prong tip 126.

In some examples the head 102 has a head diameter 110 at a widest portion of the head 102, the implant 100 has first prong length 128 from the head 102 to the first prong tip 120, the implant 100 has a second prong length 130 from the head 102 to the second prong tip 126, the implant 100 has a ratio of the first prong length 128 to the head diameter 110 of 2.0 to 10, and the implant 100 has a ratio of the second prong length 130 to the head diameter 110 of 2.0 to 10.

In some examples the implant 100 has a first prong length 128 of 8 to 40 mm from the head 102 to the first prong tip 120, and the implant 100 has a second prong length 130 of 8 to 40 mm from the head 102 to the second prong tip 126.

As noted, the first prong 112 has a first prong outer surface 116. The first prong outer surface 116 can be defined by an edge 132. For example, the edge 132 can be a single continuous edge that defines the first prong outer surface 116. Also for example, the edge 132 can be two edges that are discontinuous with respect to each other that together define the first prong outer surface 116. The edge 132 can be sharp, although other rounded, angular, smooth, and/or irregular edges may be used in further examples.

The first prong outer surface 116 can be porous, e.g. including pores having diameters of, e.g. 1 to 900 μm, 100 to 800 μm, or 200 to 600 μm, or the first prong outer surface 116 can be non-porous.

Also as noted, the second prong 114 has a second prong outer surface 122. Like for the first prong outer surface 116, the second prong outer surface 122 can be defined by an edge 134, e.g. a single continuous edge, or two edges that are discontinuous with respect to each other. The edge 134 can be sharp, although other rounded, angular, smooth, and/or irregular edges may be used in further examples. Also, the second prong outer surface 122 can be porous, e.g. including pores having diameters of, e.g. 1 to 900 μm, 100 to 800 μm, or 200 to 600 μm or the second prong outer surface 122 can be non-porous.

As noted, the first prong 112 also has a first prong inner surface 118, and the second prong 114 has a second prong inner surface 124. Like the first prong outer surface 116 and second prong outer surface 122, the first prong inner surface 118 and the second prong inner surface 124 can be defined by an edge 136 and an edge 138, respectively, e.g. a single continuous edge, or two edges that are discontinuous with respect to each other. Also, the edge 136 and the edge 138 can be sharp, although other rounded, angular, smooth, and/or irregular edges may be used in further examples.

The implant 100 also comprises first pillars 140 for contacting a hard tissue. The hard tissue can be selected from, for example, bones such as humerus, patella, tibia, or femur, among other hard tissues. In some examples the first pillars 140 may contact a hard tissue immediately upon implantation, e.g. based on extending distally from the first prong outer surface 116 and second prong outer surface 122. In some examples the first pillars 140 may contact a hard tissue over time after implantation, e.g. based on remodeling and growth of a hard tissue to come in contact with first pillars 140 over time after implantation.

The first pillars 140 are distributed on the first prong outer surface 116 and the second prong outer surface 122 across an area of at least 20 mm² on each of the first prong outer surface 116 and the second prong outer surface 122. For example, the first pillars 140 can be distributed in a regular pattern on the first prong outer surface 116 and the second prong outer surface 122, across the area of both. In this regard, the first pillars 140 can be distributed in even rows along the first prong outer surface 116 and the second prong outer surface 122, and can be distributed along a given row uniformly with respect to the distances between the centers of the first pillars 140 in the row. Also for example, the first pillars 140 can also be distributed in other regular patterns, e.g. the first pillars 140 can be distributed in rows that are even, but without the first pillars 140 being distributed uniformly within rows, the first pillars 140 in one row may be offset from the first pillars 140 in adjacent rows, the first pillars 140 may be arranged in a spiral pattern, etc. Also for example, the first pillars 140 can be distributed on the first surface in irregular patterns or randomly. For example, the first pillars can be distributed on the first prong outer surface 116 and the second prong outer surface 122 such that the first pillars 140 are packed more densely on one area of the first prong outer surface 116 or the second prong outer surface 122 and less densely on another area of the first prong outer surface 116 and the second prong outer surface 122.

The first pillars 140 can be distributed on the first prong outer surface 116 and the second prong outer surface such that none of the first pillars 140 are located at edges 132 or 134, i.e. the first prong outer surface 116 and the second prong outer surface 122 can have peripheral borders that are not occupied by any first pillars 140, resulting in the areas of the first prong outer surface 116 and the second prong outer surface 122 across which the first pillars 140 are distributed being less than the total areas of the first prong outer surface 116 and the second prong outer surface 122. In other examples the first pillars 140 can be distributed on the first prong outer surface 116 and the second prong outer surface 122 such that at least some of the first pillars 140 are located at edges 132 and/or 134, e.g. the areas of the first prong outer surface 116 and the second prong outer surface 122 across which the first pillars 140 are distributed can be equal to the total areas of the first prong outer surface 116 and the second prong outer surface 122.

The first pillars 140 extend distally from the first prong outer surface 116 and the second prong outer surface 122. In some examples all first pillars 140 extend in a uniform direction. In some examples all first pillars 140 extend distally at the same angle with respect to the first prong outer surface 116 and the second prong outer surface 122. Also for example, some first pillars 140 may extend distally at a different angle and/or in a different direction relative to other first pillars 140. In some examples the first pillars 140 extend perpendicularly from the first prong outer surface 116 and the second prong outer surface 122. This can simplify manufacturing of the implant 100. In some examples the first pillars 140 are angled toward the head of the implant 100. This can increase stability of the implant 100 following implantation in a hard tissue, e.g. an implant 100 including first pillars 140 angled this way can resist pull-out. In some examples the first pillars 140 extend from the first prong outer surface 116 and the second prong outer surface 122 at other angles and/or varying angles.

Each first pillar 140 is integral to the first prong 112 or the second prong 114, i.e. the first pillars 140, the first prong 112, and the second prong 114 are made from the same starting material, rather than, for example, the first pillars 140 being an add-on to the first prong 112 or the second prong 114. The first pillars 140 can be porous, e.g. including pores having diameters of, e.g. 1 to 900 μm, 100 to 800 μm, or 200 to 600 μm, or the first pillars 140 can be non-porous.

Each first pillar 140 has a distal end 142, corresponding to the distal-most portion of the first pillar 140 relative to the first prong outer surface 116 or the second prong outer surface 122. Each first pillar 140 can have distal edges, corresponding to edges defining the distal end 142 of each first pillar 140. Each first pillar 140 can also have lateral edges, corresponding to edges of the lateral sides of each first pillar 140. The distal edges and/or the lateral edges can be sharp, although other rounded, angular, smooth, and/or irregular edges may be used in further examples. The distal ends 142 can be flat, slanted, curved, or pointed, among other contours.

With respect to dimensions of the first pillars 140, each first pillar 140 has a transverse area, i.e. an area of a cross-section taken relative to the vertical axis along which the first pillar 140 extends distally from the first prong outer surface 116 or the second prong outer surface 122, of (100×100) to (2,000×2,000) μm². Each first pillar 140 can have a transverse area of, for example, (200 μm×200 μm) to (1,000 μm×1,000 μm), (250 μm×250 μm) to (1,000 μm×1,000 μm), (300 μm×300 μm) to (500 μm×500 μm), (350 μm×350 μm) to (450 μm×450 μm), or (395 μm×395 μm) to (405 μm×405 μm). Each first pillar 140 has a pillar height, i.e. the height of the first pillar 140 from the first prong outer surface 116 or the second prong outer surface 122 to the distal end 142 of the first pillar 140, of 100 to 2,000 μm. Each first pillar 140 can have a pillar height of, for example, 200 to 900 μm, 300 to 800 μm, or 400 to 600 μm. Each first pillar 140 has a volume, i.e. product of pillar transverse area and pillar height, of, for example (100 μm×100 μm×100 μm) to (2,000 μm×2,000 μm×2,000 μm), i.e. 1.0×10⁶ μm³ to 8×10⁹ μm³, among other volumes. The first pillars 140 extending from the first prong outer surface 116 or the second prong outer surface 122 can, for example, all have identical dimensions, e.g. identical pillar transverse areas, pillars heights, and thus identical individual volumes. Alternatively, one or more first pillars 140 can have dimensions that differ from those of other first pillars 140, such that the pillar transverse areas and/or pillar heights, and thus volumes, of the one or more first pillars 140 differ from those of the other first pillars 140.

The first pillars 140 can have, as seen from a top view, a square shape, a rectangular shape, a herringbone shape, a circular shape, or an oval shape, or alternatively can have other polygonal, curvilinear, or variable shapes. In some examples all first pillars 140 can have the same shape, e.g. a square shape, a rectangular shape, a herringbone shape, a circular shape, or an oval shape, as seen from a top view. In some examples not all first pillars 140 have the same shape as seen from a top view.

The implant 100 also comprises first slots 144 to be occupied by the hard tissue. For example, upon implantation of the implant 100 into a hard tissue, the hard tissue can immediately occupy all or part of the space corresponding to the first slots 144. This can be accomplished, for example, by pressing the implant 100 into the hard tissue. Moreover, to the extent that the hard tissue does not, upon implantation, immediately occupy all of the space corresponding to first slots 144, the hard tissue can eventually occupy all or part of the space corresponding to the first slots 144 based on remodeling and/or growth of the hard tissue over time, e.g. during healing.

The first slots 144 are defined by the first pillars 140, i.e. the first slots 144 are the spaces between the first pillars 140. Accordingly, the first slots 144 have a slot height as defined by the first pillars 140, of, for example, 100 to 2,000 μm, 200 to 900 μm, 300 to 800 μm, or 400 to 600 μm. Each first slot 144 has a width of 100 to 2,000 μm as measured along the shortest distance between adjacent first pillars 140. The first slot width can be, for example, 150 to 1,000 μm, 200 to 700 μm, or 300 to 500 μm. The first slots 144 have a volume corresponding to the volume of the space between the first pillars 140.

The implant 100 also comprises second pillars 146 for contacting a tendon or a ligament. The tendon or the ligament can be selected from among any tendon or ligament suitable for use as a tendon graft or a ligament graft. In some examples the second pillars 146 may contact a tendon or a ligament based on pulling the tendon or the ligament through the aperture 108 of the head 102. In some examples the second pillars 146 may contact a tendon or a ligament based on piercing the tendon or the ligament with the second pillars 146 prior to implantation of the implant 100, such that the tendon or the ligament becomes attached to the second pillars 146 and fixed in place with respect to the implant 100. In some examples the second pillars 146 may contact a tendon or a ligament based on pressing the tendon or the ligament against the second pillars 146, and the tendon or the ligament may be attached to the second pillars 146 indirectly, e.g. by use of a suture to fix the tendon or ligament in place with respect to the implant 100.

The second pillars 146 are distributed on the first prong inner surface 118 and the second prong inner surface 124 across an area of at least 50 mm² of each. The second pillars 146 can be distributed on the first prong inner surface 118 and the second prong inner surface 124 as described above regarding distribution of the first pillars 140 on the first prong outer surface 116 and the second prong outer surface 122, e.g. in a regular pattern, in even rows, in other regular patterns, or in irregular patterns or randomly. For example, the second pillars 146 can be distributed on the first prong inner surface 118 and the second prong inner surface 124 such that the second pillars 146 are packed more densely on one area of each of the first prong inner surface 118 and the second prong inner surface 124, e.g. near the first prong tip 120 and the second prong tip 126, respectively, and less densely on another area of each of the first prong inner surface 118 and the second prong inner surface 124, e.g. near the head 102 of the implant 100. This may be advantageous for securing the tendon or ligament to the implant 100 sufficiently while minimizing piercing of the tendon or ligament by the second pillars 132 and thus minimizing potential trauma to the tendon or ligament associated with the piercing. Thus, in some examples the second pillars 146 are distributed entirely within areas of each of the first prong inner surface 118 and the second prong inner surface 124 that are near the first prong tip 120 and the second prong tip 126, e.g. the 50%, 40%, 30%, or 20% of the areas of the first prong inner surface 118 and the second prong inner surface 124 that are closest to the first prong tip 120 and the second prong tip 126, respectively. Also like for the first pillars 140, some of the second pillars 146 can be located at edges 136 or 138, or not.

The second pillars 146 extend distally from the first prong inner surface 118 and the second prong inner surface 124. In some examples all second pillars 146 extend distally at the same angle with respect to the first prong inner surface 118 and the second prong inner surface 124. Also for example, some second pillars 146 may extend distally at a different angle and/or in a different direction relative to other second pillars 146. In some examples the second pillars 146 extend perpendicularly from the first prong inner surface 118 and the second prong inner surface 124. This can simplify manufacturing of the implant 100. In some examples the second pillars 146 on the first prong inner surface 118 are angled toward the first prong tip 120 and the second pillars 146 on the second prong inner surface 124 are angled toward the second prong tip 126. This can increase stability of a tendon or a ligament attached to the implant 100 following implantation in a hard tissue, e.g. by decreasing the risk of separation of the tendon or ligament from the implant 100. In some examples the second pillars 146 extend from the first prong inner surface 118 and the second prong inner surface 124 at other angles and/or varying angles.

Like each first pillar 140, each second pillar 146 is integral to the first prong 112 or the second prong 114, i.e. the second pillars 146, the first prong 112, and the second prong 114 are made from the same starting material, rather than, for example, the second pillars 146 being an add-on to the first prong 112 or the second prong 114. The second pillars 146 can be porous, e.g. including pores having diameters of, e.g. 1 to 900 μm, 100 to 800 μm, or 200 to 600 μm, or the second pillars 146 can be non-porous.

Also like each first pillar 140, each second pillar 146 has a distal end 148, corresponding to the distal-most portion of the second pillar 146 relative to the first prong inner surface 118 and the second prong inner surface 124. Each second pillar 146 can have distal edges, corresponding to edges defining the distal end 148 of each second pillar 146. Each second pillar 146 can also have lateral edges, corresponding to edges of the lateral sides of each second pillar 146. The distal edges and/or the lateral edges can be sharp, although other rounded, angular, smooth, and/or irregular edges may be used in further examples. The distal ends 148 can be flat, slanted, curved, or pointed, among other contours.

With respect to dimensions of the second pillars 146, each second pillar 146 has a transverse area, i.e. an area of a cross-section taken relative to the vertical axis along which the second pillar 146 extends distally from the first prong inner surface 118 or the second prong inner surface 124, of (200×200) to (4,000×4,000). Each second pillar 146 can have a transverse area of, for example, (400 μm×400 μm) to (2,000 μm×2,000 μm), or (1,000 μm×1,000 μm) to (2,000 μm×2,000 μm). Each second pillar 146 has a pillar height, i.e. the height of the second pillar 146 from the first prong inner surface 118 or the second prong inner surface 124 to the distal end 148 of the second pillar 146, of 100 to 5,000 μm. Each second pillar 146 can have a pillar height of, for example, 200 to 4,000 μm, 300 to 3,000 μm, 400 to 2,000 μm, or 500 to 1,000 μm. Each second pillar 146 has a volume, i.e. product of pillar transverse area and pillar height, of, for example (200 μm×200 μm×100 μm) to (4,000 μm×4,000 μm×5,000 μm), i.e. 4.0×10⁶ μm³ to 8.0×10¹⁰ μm³, among other volumes. The second pillars 146 extending from the first prong inner surface 118 and the second prong inner surface 124 can, for example, all have identical dimensions, e.g. identical pillar transverse areas, pillars heights, and thus identical individual volumes. Alternatively, one or more second pillars 146 can have dimensions that differ from those of other second pillars 146, such that the pillar transverse areas and/or pillar heights, and thus volumes, of the one or more second pillars 146 differ from those of the other second pillars 146.

Like the first pillars 140, the second pillars 146 can have, as seen from a top view, a square shape, a rectangular shape, a herringbone shape, a circular shape, or an oval shape, or alternatively can have other polygonal, curvilinear, or variable shapes.

The implant 100 also comprises second slots 150 to be occupied by the tendon or the ligament. For example, upon the second pillars 146 contacting the tendon or the ligament, the tendon or the ligament can occupy most or all of the space corresponding to the second slots 150. This can be accomplished, for example, by matching the width of the tendon or the implant to a width of the first prong inner surface 118 or the second prong inner surface 124, such that the tendon or the ligaments fits on or across the second pillars 146 and within the second slots 150.

The second slots 150 are defined by the second pillars 146, similarly as the first slots 144 are defined by the first pillars 140. Accordingly, the second slots 150 have a slot height as defined by the second pillars 146, of, for example, 100 to 5,000 μm, 200 to 4,000 μm, 300 to 3,000 μm, 400 to 2,000 μm, or 500 to 1,000 μm. Each second slot 150 has a width of 400 to 4,000 μm as measured along the shortest distance between adjacent second pillars 146. The second slot width can be, for example, 500 to 3,000 μm, 600 to 2,000 μm, or 800 to 1,500 μm. The second slots 150 have a volume corresponding to the volume of the space between the second pillars 146.

The implant 100 has a ratio of (i) the sum of the volumes of the first slots 144 to (ii) the sum of the volumes of the first pillars 140 and the volumes of the first slots 144 (“first surface ratio”) of 0.40:1 to 0.90:1. The first surface ratio can be, for example, 0.51:1 to 0.90:1, 0.51:1 to 0.60:1, or 0.70:1 to 0.76:1.

The implant 100 also has a ratio of (i) the sum of the volumes of the second slots 150 to (ii) the sum of the volumes of the second pillars 146 and the volumes of the second slots 150 (“second surface ratio”) of 0.60:1 to 0.98:1. The second surface ratio can be, for example, 0.71:1 to 0.98:1, 0.75:1 to 0.97:1, 0.80:1 to 0.96:1, 0.80:1 to 0.90:1, 0.85:1 to 0.95:1, about 0.80:1, about 0.85:1, about 0.90:1, or about 0.95:1.

The second surface ratio is greater than the first surface ratio.

Without wishing to be bound by theory, it is believed that the first surface ratio determines the approximate percentages of hard tissue and implant 100 that will occupy an outer surface interface following implantation of the implant 100, e.g. that upon inserting the implant 100 into the hard tissue, or upon remodeling and growth of the hard-tissue following implantation, that the hard tissue will occupy all or essentially all of the space corresponding to the first slots 144 of the implant 100. The outer surface interface includes (i) the first pillars 140, (ii) the first slots 144, which, upon or following implantation, become occupied by hard tissue, (iii) any additional space between the first prong outer surface 116 and the second prong outer surface 122 and a curved surface defined by the distal ends 142 of the first pillars 140, e.g. the space between peripheral borders of the first prong outer surface 116 and the second prong outer surface 122 that is not occupied by first pillars 140 and the curved surface, which also becomes occupied by hard tissue, and (iv) any pores on the first prong outer surface 116, the second prong outer surface 122, and/or the first pillars 140, which, depending on their size, may also become occupied by hard tissue. Accordingly, for example, a first surface ratio of 0.40:1 would, following implantation of an implant 100 and subsequent remodeling and growth of hard tissue, wherein the implant 100 includes edges 132 and 134 around the first prong outer surface 116 and the second prong outer surface 122, respectively, and for which first pillars 140 are located at the edges 132 and/or 134, result in an interface that includes by volume 40% hard tissue and 60% implant 100, and more particularly 60% first pillars 140 of the implant 100. Similarly, a first surface ratio of 0.40:1 would, following implantation of an implant 100 and subsequent remodeling and growth of hard tissue, wherein the implant 100 includes edges 132 and 134 around the first prong outer surface 116 and the second prong outer surface 122 and for which no first pillars 140 are located at the edges 132 and/or 134, result in an interface that includes by volume more than 40% hard tissue and less than 60% implant 100, with the percentage of hard tissue increasing, and the percentage of implant 100 decreasing, with increasing distance between the peripheral-most first pillars 140 and first slots 144 and the edges 132 and 134 around the first prong outer surface 116 and the second prong outer surface 122. By way of further examples, first surface ratios of 0.51:1, 0.60:1, 0.70:1, 0.76:1, and 0.90:1, would result in outer surface interfaces that include, by volume, 51% hard tissue and 49% implant 100, 60% hard tissue and 40% implant 100, 70% hard tissue and 30% implant 100, 76% hard tissue and 24% implant 100, and 90% hard tissue and 10% implant, respectively, for an implant 100 wherein the implant 100 includes edges 132 and 134 around the first prong outer surface 116 and the second prong outer surface 122 and for which first pillars 140 are located at the edges 132 and/or 134. Moreover, the percentage of hard tissue would increase, and the percentage of implant 100 would decrease, with increasing distance between the peripheral-most first pillars 140 and first slots 144 and the edges 132 and 134 of the first prong outer surface 116 and the second prong outer surface 122. It is believed that by achieving an outer surface interface that is at least 40% hard tissue, but that has a sufficient amount of the implant 100 to provide support and to keep the implant 100 from migrating, the outer surface interface will exhibit properties similar to those of the bulk hard tissue adjacent to the interface, e.g. high resilience to load.

Without wishing to be bound by theory, it also is believed that the second surface ratio determines the approximate percentages of tendon or ligament and implant 100 that will occupy an inner surface interface following implantation of the implant 100, e.g. that upon attaching the tendon or the ligament to the implant 100 such that the tendon or the ligament is in contact with the second pillars 146, and upon inserting the implant 100 into the hard tissue, that the tendon or the ligament will occupy all or essentially all of the space corresponding to the second slots 150 of the implant 100. The inner surface interface includes (i) the second pillars 146, (ii) the second slots 150, which, upon attachment of the tendon or ligament, become occupied by the tendon or ligament, (iii) any additional space between the first prong inner surface 118 and the second prong inner surface 124 and a curved surface defined by the distal ends 148 of the second pillars 146, e.g. the space between a peripheral border of the first prong inner surface 118 and the second prong inner surface 124 that is not occupied by second pillars 146 and the curved surface, which also becomes occupied by the tendon or ligament, and (iv) any pores on the first prong inner surface 118, the second prong inner surface 124, and/or the second pillars 146, which, depending on their size, may also become occupied by the tendon or ligament. It is believed that use of an implant 100 for which the second surface ratio is greater than the first surface ratio will provide a sufficient surface for attachment of a tendon or ligament, corresponding to the first prong inner surface 118 and the second prong inner surface 124 including the second pillars 146, e.g. based on piercing of the tendon or ligament by the second pillars 146 in a discrete area of limited size, while protecting the tendon or ligament upon placement of the implant 100 in a hard tissue, by minimizing further trauma to the tendon or ligament during implantation, e.g. the tendon or ligament can be attached to the implant 100 such that most or all of the tendon or ligament fits between the first prong inner surface 118 and the second prong inner surface 124 and the distal ends 148 of the second pillars 146, such that the tendon or ligament is subject to little or no contact with hard tissue during insertion of the implant 100 into the hard tissue and no screw or other fixation device is subsequently driven through the tendon or ligament following the insertion. It is believed that this will promote hard-tissue remodeling and growth of the hard tissue at the site of implantation, and hold the tendon or ligament stable following implantation to allow formation of a fibrovascular scar tissue between the hard tissue and the tendon or ligament that can effectively transfer stress from tendon or ligament to bone and vice versa.

Considering additional features of the implant 100 for attaching a tendon or a ligament to a hard tissue in more detail, in some examples that implant 100 comprises one or more additional prongs, e.g. a third prong, a fourth prong, etc. Like the first prong 112 and the second prong 114, each additional prong extends from the lower surface 106 of the head 102 and has a prong outer surface, a prong inner surface, a prong tip, first pillars, first slots, second pillars, and second slots, as described above for the first prong 112 and the second prong 114.

In some examples the implant 100 further comprises a tool-engaging portion. In some examples the tool-engaging portion comprises a recess in the head 102 of the implant 100. In these examples, the tool-engaging portion, such as the recess, can be used for engagement of the implant 100 by a tool for pressing, driving, or otherwise inserting the implant 100 into a hard tissue.

In some examples, the implant 100 further comprises one or more holes at or near the first prong tip 120 and/or the second prong tip 126. The holes can be used for passing a suture. The suture can then be used for pulling the implant 100 into a hard tissue, e.g. into a bone tunnel of a hard tissue.

The implant 100 can be made by fabrication methods such as laser cutting, injection molding, or 3D printing, among others.

Methods of Using Implants for Attaching a Tendon or a Ligament to a Hard Tissue

Methods will now be described for use of the implant 100 for attaching a tendon or a ligament to a hard tissue in an individual in need thereof. The implant 100 is as described above.

The method includes a step of (1) preparing a bone tunnel in a bone of the individual. The preparing of the bone tunnel can comprise, for example, drilling a hole in the bone. The preparing of the bone tunnel also can comprise, for example, tapping a hole to provide a thread.

The method also includes a step of (2) attaching a tendon or a ligament to the implant 100 such that the tendon or ligament is in contact with the second pillars 146 of the implant 100. Residual muscle can be removed from the tendon or ligament prior to attaching the tendon or ligament to the implant 100 as needed. In some examples the attaching of the tendon or the ligament to the implant 100 comprises pulling the tendon or the ligament through the aperture 108, thereby putting the tendon or ligament in contact with the second pillars 146 of the implant 100. In some examples the attaching comprises piercing the tendon or the ligament with the second pillars 146, thereby putting the tendon or ligament in contact with the second pillars 146 of the implant 100. In some examples the attaching comprises use of a suture or an adhesive to attach the tendon or the ligament to the implant 100. In some examples the method does not comprise use of a suture or an adhesive to attach the tendon or the ligament to the implant 100. For example, in some examples the piercing can be sufficient for attaching the tendon or ligament to the implant 100, thus simplifying the attachment.

The method also includes a step of (3) inserting the implant 100 into the bone tunnel. In some examples the inserting of the implant 100 into the bone tunnel comprises pressing the implant 100 into the bone tunnel. In some examples the inserting of the implant 100 into the bone tunnel comprises pulling the implant 100 into the bone tunnel. In some examples the inserting of the implant 100 into the bone tunnel comprises driving the implant 100 into the bone tunnel by rotating the implant 100.

As noted above, the first pillars 140 may be pressed into the hard tissue, potentially eliminating micro-motion and migration of the implant 100 over time, accommodating torque, and/or eliminating the need for adhesives such as cement or grout to hold the implant 100 in place. Accordingly, in some examples, the inserting of the implant 100 can be done without use of screws or plating mechanisms. This can minimize the number and profiles of implants used in the method in an individual while still eliminating micro-motion and migration of the hard-tissue implant 100 over time. Also, in some examples, the inserting of the implant 100 can be done without use of adhesives, e.g. cement or grout. This can simplify the method while still eliminating micro-motion and migration of the hard-tissue implant 100 over time.

In some examples the inserting of the implant 100 comprises having the first pillars 140 penetrate the hard tissue, partially or completely. This can be accomplished, for example, by preparing the bone tunnel to have a diameter greater than or equal to a distance 152 between the first prong outer surface 116 and the second prong outer surface 122, but less than a distance 154 between the distal ends 142 of the first pillars 140 on the first prong outer surface 116 and the distal ends 142 of the first pillars 140 on the second prong outer surface 122. For example, the implant 100 can be pressed, inserted, or driven into the bone tunnel such that the first pillars 140 penetrate bone of the bone tunnel to a depth of, for example, 100 to 2,000 μm, 200 to 900 μm, 300 to 800 μm, or 400 to 600 μm. Also for example, the implant 100 can be pressed, inserted, or driven into the bone tunnel such that first pillars 140 penetrate bone of the bone tunnel to a depth, relative to the height of the first pillars 140, of for example 25%, 50%, 75%, and 100% of the height of the first pillars 140. In some of these examples the inserting of the implant 100 comprises pressing or pulling the implant 100 into the bone tunnel, then rotating the implant 100 slightly. This can cause the first pillars 140 to bite into bone between the first pillars 140, resulting in the implant 100 becoming locked in place in the bone.

The method results in attaching the tendon or the ligament to the bone of the individual.

In some examples additional hard tissue can be added to the first prong outer surface 116, the second prong outer surface 122, and/or the first pillars 140 of the implant 100 prior to implanting. For example, shavings of hard-tissue of a patient, generated during preparation work including sawing or drilling of hard tissue of the patient, can be added. This may promote growth of hard tissue into the first slots 144 of the implant 100 following implantation.

Also in some examples additional compositions can be added to the first prong outer surface 116, the second prong outer surface 122, and/or the first pillars 140 of the implant 100 prior to implanting. Such compositions include, for example, blood, one or more antibiotics, one or more osteogenic compounds, bone marrow aspirate, and/or surface chemistry for inducing early bone ingrowth. For example, the first prong outer surface 116, the second prong outer surface 122, and/or the first pillars 140 of the implant 100 can be coated with one or more such compositions, with the first pillars 140 retaining the compositions during implantation. This also may promote growth of tissue into the first slots 144 of the implant 100 following implantation.

Standard approaches for implanting the implant 100, e.g. pressing the implant 100 into the bone tunnel in the bone, are known in the art and can be used in the methods disclosed here.

The hard tissue can be selected from, for example, bones such as humerus, patella, tibia, or femur, among other hard tissues, as discussed above. In some examples the first pillars 140 may contact a hard tissue immediately upon implantation, e.g. based on extending distally from the first prong outer surface 116 and the second prong outer surface 122. In some examples the first pillars 140 may contact a hard tissue over time after implantation, e.g. based on remodeling and growth of a hard tissue to come in contact with first pillars 140 for which distal ends 142 of the first pillars 140 are recessed relative to a surrounding surface of the implant 100.

The method can be applied to examples of the implant 100 as disclosed above. The first surface ratio and the second surface ratio can be determined as discussed above.

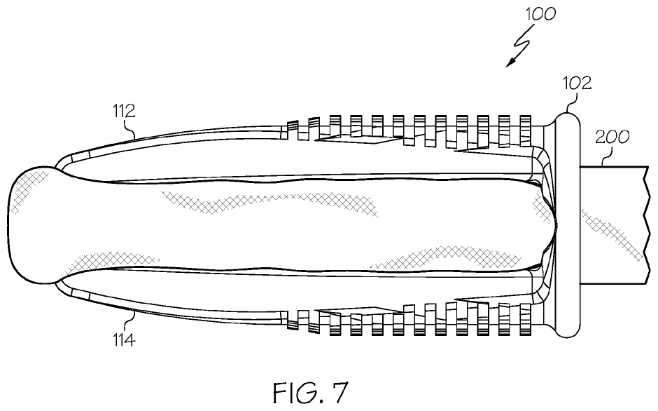

FIG. 7 shows a side view of the implant 100 to which a tendon 200 has been pressed onto second pillars 146 of the implant 100. The tendon 200 has been passed through the aperture 108 in the head 102 of the implant 100 and attached to the implant 100 at the second pillars 146. A ligament could similarly be attached to the implant 100.

It will be apparent to those skilled in the art that various modifications and variations can be made without departing from the spirit and scope of the claimed invention.

1. An implant for attaching a tendon or a ligament to a hard tissue comprising:
(a) a head having an upper surface, a lower surface, and an aperture between the upper surface and the lower surface;
(b) a first prong extending from the lower surface of the head, and having a first prong outer surface, a first prong inner surface, and a first prong tip;
(c) a second prong extending from the lower surface of the head, and having a second prong outer surface, a second prong inner surface, and second prong tip;
(d) first pillars for contacting a hard tissue, the first pillars being distributed on the first prong outer surface and the second prong outer surface across an area of at least 20 mm² on each, and extending distally therefrom, and each first pillar being integral to the first prong or the second prong, having a distal end, having a transverse area of (100×100) to (2,000×2,000) μm², and having a height of 100 to 2,000 μm;
(e) first slots to be occupied by the hard tissue, the first slots being defined by the first pillars and each first slot having a width of 100 to 2,000 μm as measured along the shortest distance between adjacent first pillars;
(f) second pillars for contacting a tendon or a ligament, the second pillars being distributed on the first prong inner surface and the second prong inner surface across an area of at least 20 mm² of each, and extending distally therefrom, and each second pillar being integral to the first prong or the second prong, having a distal end, having a transverse area of (200×200) to (4,000×4,000)μm², and having a height of 100 to 5,000 μm; and
(g) second slots to be occupied by the tendon or the ligament, the second slots being defined by the second pillars and each second slot having a width of 400 to 4,000 μm as measured along the shortest distance between adjacent second pillars; wherein:
the implant has (1) a Young's modulus of elasticity of at least 3 GPa, (2) a ratio of (i) the sum of the volumes of the first slots to (ii) the sum of the volumes of the first pillars and the volumes of the first slots (“first surface ratio”) of 0.40:1 to 0.90:1, and (3) a ratio of (i) the sum of the volumes of the second slots to (ii) the sum of the volumes of the second pillars and the volumes of the second slots (“second surface ratio”) of 0.60:1 to 0.98:1; and
the second surface ratio is greater than the first surface ratio.

2. The implant of claim 1, wherein the implant is made of one or more materials selected from implantable-grade polyaryletherketone that is essentially unfilled, implantable-grade polyetheretherketone, implantable-grade polyetherketoneketone, titanium, stainless steel, cobalt-chromium alloy, titanium alloy, Ti-6Al-4V titanium alloy, Ti-6Al-7Nb titanium alloy, ceramic material, silicon nitride (Si3N4), implantable-grade composite material, implantable-grade polyaryletherketone with filler, implantable-grade polyetheretherketone with filler, implantable-grade polyetheretherketone with carbon fiber, or implantable-grade polyetheretherketone with hydroxyapatite.

3-4. (canceled)

5. The implant of claim 1, wherein the first prong and the second prong extend perpendicularly from the lower surface of the head.

6. The implant of claim 1, wherein the first prong is tapered toward the first prong tip, and the second prong is tapered toward the second prong tip.

7. (canceled)

8. The implant of claim 1, wherein the first pillars are perpendicular to the first prong outer surface and the second prong outer surface.

9. The implant of claim 1, wherein the first pillars are angled toward the head.

10. The implant of claim 1, wherein the second pillars extend in a uniform direction.

11. The implant of claim 1, wherein the second pillars on the first prong inner surface are angled toward the first prong tip and the second pillars on the second prong inner surface are angled toward the second prong tip.

12. The implant of claim 1, wherein the transverse area of each first pillar is (200×200) μm² to (1,000×1,000) μm².

13. The implant of claim 1, wherein the height of each first pillar is 200 to 900 μm.

14. (canceled)

15. The implant of claim 1, wherein the width of each first slot is 200 to 900 μm.

16. The implant of claim 1, wherein the transverse area of each second pillar is (400×400) μm² to (2,000×2,000) μm².

17. The implant of claim 1, wherein the height of each second pillar is 200 to 4,000 μm.

18. (canceled)

19. The implant of claim 1, wherein the width of each second slot is 500 to 3,000 μm.

20. The implant of claim 1, wherein the head has a head diameter at a widest portion of the head, the implant has first prong length from the head to the first prong tip, the implant has a second prong length from the head to the second prong tip, the implant has a ratio of the first prong length to the head diameter of 2.0 to 10, and the implant has a ratio of the second prong length to the head diameter of 2.0 to 10.

21. The implant of claim 1, wherein the head has a head diameter of 4 to 20 mm at a widest portion of the head.

22. The implant of claim 1, wherein the implant has a first prong length of 8 to 40 mm from the head to the first prong tip, and the implant has a second prong length of 8 to 40 mm from the head to the second prong tip.

23. The implant of claim 1, wherein one or more of the head, the first prong, the first pillars, the second prong, or the second pillars are non-porous.

24. The implant of claim 1, wherein one or more of the head, the first prong, the first pillars, the second prong, or the second pillars are porous.

25. A method of use of the implant of claim 1 for attaching a tendon or a ligament to a hard tissue in an individual in need thereof, the method comprising steps of:
(1) preparing a bone tunnel in a bone of the individual;
(2) attaching a tendon or a ligament to the implant such that the tendon or ligament is in contact with the second pillars of the implant; and
(3) inserting the implant into the bone tunnel;
thereby attaching the tendon or the ligament to the bone of the individual.

26-30. (canceled)
